(19) United States
(12) Patent Application Publication
(10) Pub. No.: US 2018/0368695 A1
(43) Pub. Date: Dec. 27, 2018

(54) APPARATUS, METHOD, AND PROGRAM OF ACQUIRING OPTICAL COEFFICIENT INFORMATION

(71) Applicant: CANON KABUSHIKI KAISHA, Tokyo (JP)

(72) Inventor: Yoshiko NAKAMURA, Kusatsu-shi (JP)

(21) Appl. No.: 15/741,398

(22) PCT Filed: Jun. 29, 2016
(86) PCT No.: PCT/JP2016/003119

§ 371 Date: Jan. 2, 2018

(30) Foreign Application Priority Data
Jul. 6, 2015 (JP) 2015-135672

(51) Int. Cl. A61B 5/00; G01N 29/07; G01N 21/17; G01N 29/24

(52) U.S. Cl. CPC A61B 5/0095; G01N 29/07; A61B 5/708; G01N 29/2418; A61B 5/4312; G01N 21/17

(57) ABSTRACT

An apparatus according to the present invention includes a first acquiring unit configured to acquire information indicative of a relationship between sound speed information and optical coefficient information; a second acquiring unit configured to acquire sound speed information of a specimen; and a third acquiring unit configured to acquire optical coefficient information of the specimen by using the sound speed information of the specimen and the information indicative of the relationship.

### TECHNICAL FIELD

The present invention relates to an apparatus, a method, and a program of acquiring optical coefficient information.

### BACKGROUND ART

There is suggested clinical application of an apparatus that estimates optical coefficient information (light absorption coefficient, reduced scattering coefficient, effective attenuation coefficient, etc.) of a specimen such as a living body. Also, as a method of measuring optical coefficient information of a specimen, for example, there are suggested diffuse optical tomography (DOT) described in NPL 1 and time-resolved spectroscopy (TRS) described in NPL 2.

### CITATION LIST

### Non Patent Literature

NPL 1: A. P. Gibson, et al., “Recent Advances in Diffuse Optical Imaging,” Phys. Med. Biol. 50 (2005), R1 to R43
NPL 2: Kazunori Suzuki, “Quantitative Measurement of Optical Parameters in Normal Breasts Using Time-resolved Spectroscopy, In Vivo Results of 30 Japanese Women,” Journal of Biomedical Optics 1 (3), 330 to 334 (July 1996)

### SUMMARY OF INVENTION

### Solution to Problem

An apparatus according to the present invention includes a first acquiring unit configured to acquire information indicative of a relationship between sound speed information and optical coefficient information; a second acquiring unit configured to acquire sound speed information of a specimen; and a third acquiring unit configured to acquire optical coefficient information of the specimen by using the sound speed information of the specimen and the information indicative of the relationship.

Further features of the present invention will become apparent from the following description of exemplary embodiments with reference to the attached drawings.

### BRIEF DESCRIPTION OF DRAWINGS

FIG. 1 is a schematic illustration showing a configuration of a photoacoustic apparatus according to a first embodiment.

FIG. 2 is an illustration showing a specific example of a computer according to the first embodiment.

FIG. 3 is a flowchart of an operation of the photoacoustic apparatus according to the first embodiment.

FIG. 4A is an illustration showing an example of a calculation result of a sound speed and an optical coefficient.

FIG. 4B is an illustration showing an example of a calculation result of the sound speed and the optical coefficient.

FIG. 5A is an illustration showing a graph of a relational expression (linear function approximate expression) between the sound speed and the optical coefficient.

FIG. 5B is an illustration showing a graph of a relational expression (linear function approximate expression) between the sound speed and the optical coefficient.

FIG. 6A is an illustration showing a graph of a relational expression (cubic function approximate expression) between the sound speed and the optical coefficient.

FIG. 6B is an illustration showing a graph of a relational expression (cubic function approximate expression) between the sound speed and the optical coefficient.

FIG. 7A is an illustration showing a graph of a relational expression (logarithmic function approximate expression) between the sound speed and the optical coefficient.

FIG. 7B is an illustration showing a graph of a relational expression (logarithmic function approximate expression) between the sound speed and the optical coefficient.

FIG. 8A is an illustration showing a graph of a relational expression (two linear function approximate expressions) between the sound speed and the optical coefficient.

FIG. 8B is an illustration showing a graph of a relational expression (two linear function approximate expressions) between the sound speed and the optical coefficient.

FIG. 9 is an illustration showing the relationship between the sound speed and the optical coefficient for each category of mammary gland density.

FIG. 10 is a flowchart of an operation of a photoacoustic apparatus according to a third embodiment.

### DESCRIPTION OF EMBODIMENTS

Desirable embodiments of the invention are described below with reference to the drawings. The dimensions, materials, and shapes of components described below, relative arrangement of the components, and so forth should be properly changed in accordance with the configuration of an apparatus to which the invention is applied and various conditions. Hence, it is not intended to limit the scope of the invention to the description given below.

### First Embodiment

In this embodiment, an example is described, in which optical coefficient information acquired by a new method is used for photoacoustic imaging (PAI).

A photoacoustic imaging apparatus irradiates a specimen with pulsed light, receives a photoacoustic wave (an ultrasound) generated as the result that a tissue in the specimen absorbs the energy of the irradiation light, and generates a generation sound pressure (initial sound pressure) distribution of the photoacoustic wave. A photoacoustic wave is generated when energy of absorbing light irradiated on a specimen is converted into a sound pressure. An initial sound pressure P₀ when a photoacoustic wave is generated can be expressed by Expression (1).

P₀(r)=Γ(r)×μₐ(r)×φ(r) (1)

In Expression (1), r represents a position in the specimen. P₀ represents an initial sound pressure, which is acquired on the basis of a reception signal of a photoacoustic wave. Γ is a Gruneisen coefficient, which is a known parameter uniquely determined when a tissue is determined. μₐ represents a light absorption coefficient. φ represents a light fluence.

Referring to Expression (1), it is understood that the light fluence of the light emitted on the specimen has to be calculated at each position of the specimen, to acquire a light absorption coefficient distribution in the specimen. For this calculation, optical coefficient information, such as a light absorption coefficient, a reduced scattering coefficient, or an effective attenuation coefficient, in the specimen is required.

Owing to this, in this embodiment, a new method of acquiring optical coefficient information of a specimen, used for photoacoustic imaging, is described. In this embodiment, optical coefficient information of a specimen is acquired on the basis of the relationship between sound speed information and optical coefficient information, found by the inventor.

Information acquired by use in photoacoustic imaging according to this embodiment is, for example, a light absorption coefficient or information relating to the concentration of a substance configuring a specimen. The information relating to the concentration of a substance is, for example, the concentration of oxyhemoglobin, the concentration of deoxyhemoglobin, the concentration of total hemoglobin, or oxygen saturation. The concentration of total hemoglobin is the sum of the concentrations of oxyhemoglobin and deoxyhemoglobin. The oxygen saturation is the ratio of oxyhemoglobin to all hemoglobin. In this embodiment, distribution information representing the value of the above-described information at each position (each position in a two-dimensional or three-dimensional space) of the specimen, and a representative value (an average or another value) of the above-described information of the specimen are acquired as specimen information.

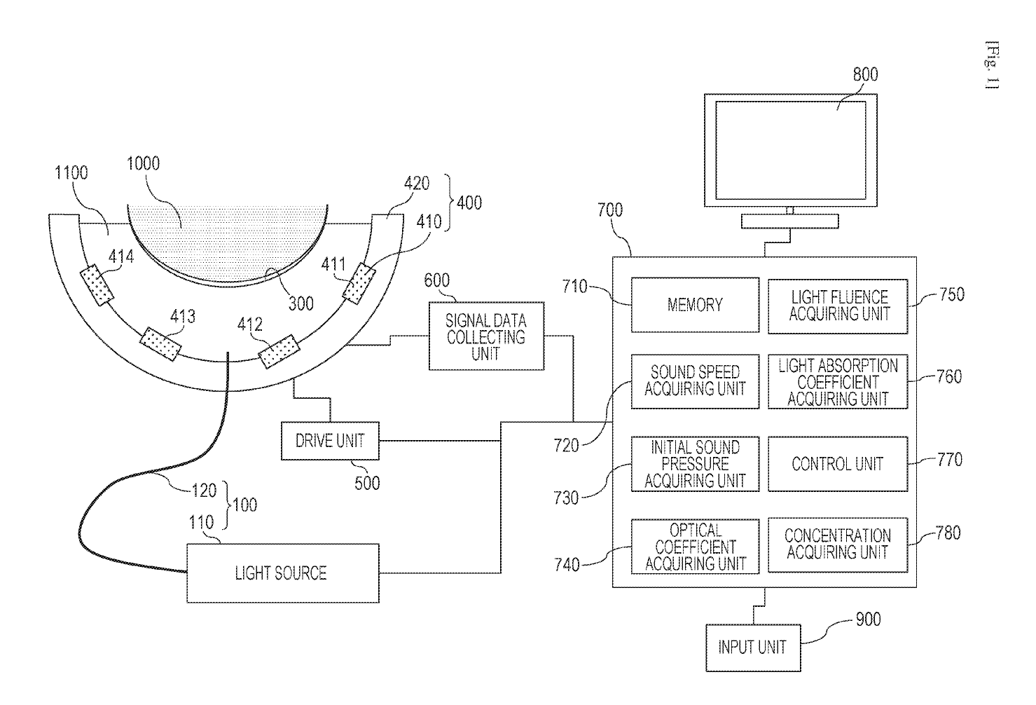

FIG. 1 is a schematic illustration showing a photoacoustic apparatus according to this embodiment. The photoacoustic apparatus includes a light irradiation unit 100, a holding unit 300, a receiving unit 400, a drive unit 500, a signal data collecting unit 600, a computer 700, a display unit 800, and an input unit 900. An object to be measured is a specimen 1000.

### Light Irradiation Unit 100

The light irradiation unit 100 includes a light source 110 that emits light 130, and an optical system 120 that guides the light 130 emitted from the light source 110 to the specimen 1000.

The light source 110 may be desirably a pulsed light source that can generate pulsed light in nanosecond to microsecond order. The pulse width of the light may be about 1 to about 100 nanoseconds. Also, the wavelength of the light may be in a range from about 400 nm to about 1600 nm. If a blood vessel near a surface of a living body is imaged with high resolution, a wavelength largely absorbed by the blood vessel (in a range from 400 nm to 700 nm) may be used. In contrast, if a deep portion of the living body is imaged, a wavelength (in a range from 700 nm to 1100 nm) typically less absorbed by a background tissue (for example, water and fat) of the living body may be used.

For the light source 110, a laser or a light-emitting diode may be used. Also, when measurement is performed by using light with a plurality of wavelengths, the light source may change the wavelength. If a specimen is irradiated with a plurality of wavelengths, a plurality of light sources that generate light with mutually different wavelengths may be prepared, and a specimen may be alternately irradiated with the light from the respective light sources. Even if the plurality of light sources are used, the light sources are collectively expressed as a light source. For the laser, any one of various layers including a solid-state laser, a gas laser, a dye laser, and a semi-conductor laser may be used. A pulse laser, such as a Nd:YAG laser or an alexandrite laser, may be desirably used. Alternatively, a Ti:sa laser or an optical parametric os-cillators (OPO) laser using Nd:YAG laser light as exciting light may be used.

The optical system 120 can use optical elements such as a lens, a mirror, an optical fiber, and so forth. When a breast or the like serves as the specimen 1000, the beam diameter of pulsed light is desirably spread and irradiated, and hence the light emitting portion of the optical system 120 may be configured of a diffusion plate or the like that diffuses light. In contrast, in a photoacoustic microscope, the light emitting portion of the optical system 120 may be configured of a lens or the like, and a beam may be focused and irradiated.

Alternatively, the light irradiation unit 100 may not include the optical system 120, and the light source 110 may directly irradiate the specimen 1000 with the light 130.

### Holding Unit 300

The holding unit 300 is used for holding the shape of the specimen during measurement. Since the holding unit 300 holds the specimen 1000, the movement of the specimen can be restricted, and the position of the specimen 1000 can be held within the holding unit 300. For the material of the holding unit 300, PET-G or the like can be used.

The holding unit 300 may be desirably formed of a material having a certain hardness that can hold the specimen 1000. The holding unit 300 may be formed of a material that transmits light used for measurement. The holding unit 300 may be formed of a material having an impedance substantially equivalent to the specimen 1000. If an object having a curved surface such as a breast serves as the specimen 1000, the holding unit 300 may be molded in a recessed shape. In this case, the specimen 1000 can be inserted into the recessed portion of the holding unit 300. However, the photoacoustic apparatus according to this embodiment may not include the holding unit 300. Also, the photoacoustic apparatus according to this embodiment may not include the holding unit 300 and may have an opening that allows a breast to be inserted.

### Receiving Unit 400

The receiving unit 400 includes a receiving element group 410 and a support body 420 that supports the receiving element group 410. The receiving element group 410 includes receiving elements 411 to 414 that receive acoustic waves and outputs electric signals.

A member that configures each of the receiving elements 411 to 414 can use a piezoelectric ceramic material represented by lead zirconate titanate (PZT), or a polymer piezoelectric film material represented by polyvinylidene fluoride (PVDF). Alternatively, an element other than a piezoelectric element may be used. For example, a capacitive transducer (capacitive micro-machined ultrasonic transducers (CMUT)), or a transducer using a Fabry-Perot interferometer may be used. It is to be noted that any transducer may be employed as a receiving element as long as the receiving element can receive an acoustic wave and output a signal.

The support body 420 may be formed of a metal material or other material having high mechanical strength. In this embodiment, the support body 420 has a hemispherical shell shape, and is configured to support the receiving element group 410 on the hemispherical shell. In this case, the directional axes of the respective receiving elements are collected at a position near the center of the curvature of the hemisphere. When imaging is performed by using an electric signal group output from these receiving elements, the image quality at the position near the center of the curvature is high. However, the support body 420 may have any configuration as long as the support body 420 can support the receiving element group 410.

### Signal Data Collecting Unit 600

The signal data collecting unit 600 includes an amplifier that amplifies an electric signal being an analog signal output from each of the receiving elements 411 to 414, and an A/D converter that converts the analog signal output from the amplifier into a digital signal. The digital signal output from the signal data collecting unit 600 is stored in a memory 710 in the computer 700. The signal data collecting unit 600 is also called data acquisition system (DAS). In this specification, an electric signal is a concept including an analog signal and a digital signal.

### Computer 700

The computer 700 includes the memory 710, a sound speed acquiring unit 720, an initial sound pressure acquiring unit 730, an optical coefficient acquiring unit 740, a light fluence acquiring unit 750, a light absorption coefficient acquiring unit 760, a control unit 770, and a concentration acquiring unit 780.

The memory 710 can be configured of a non-temporary storage medium, such as a magnetic disk or a flash memory. Alternatively, the memory 710 may be a volatile medium such as a dynamic random access memory (DRAM). It is to be noted that a storage medium storing a program is a non-temporary storage medium.

Units having arithmetic functions, such as the sound speed acquiring unit 720, the optical coefficient acquiring unit 740, the initial sound pressure acquiring unit 730, the light fluence acquiring unit 750, the light absorption coefficient acquiring unit 760, and the concentration acquiring unit 780 each can be configured of a processor being a CPU or a graphics processing unit (GPU); or an arithmetic circuit such as a field pro-grammable gate array (FPGA) chip. These units each may not be configured of a single processor or a single arithmetic circuit, and may be configured of a plurality of processors or a plurality of arithmetic circuits.

The control unit 770 is configured of an arithmetic element such as a CPU. The control unit 770 receives signals by various operations, such as start of imaging from the input unit 900, and controls respective configurations of the photoacoustic apparatus. Also, the control unit 770 reads out a program code stored in the memory 710, and controls operations of respective configurations of the photoacoustic apparatus.

The computer 700 is a device that stores a digital signal output from the signal data collecting unit 600 and acquires specimen information on the basis of the stored digital signal. Processing executed by the computer 700 will be described later in detail.

It is to be noted that respective functions of the computer 700 may be configured of different pieces of hardware. Alternatively, the receiving unit 400, the signal data collecting unit 600, and the computer 700 may be configured of a single piece of hardware. Still alternatively, at least portions of the respective configurations may be configured of a single piece of hardware. For example, the receiving unit 400 and the signal data collecting unit 600 may be configured of a single piece of hardware.

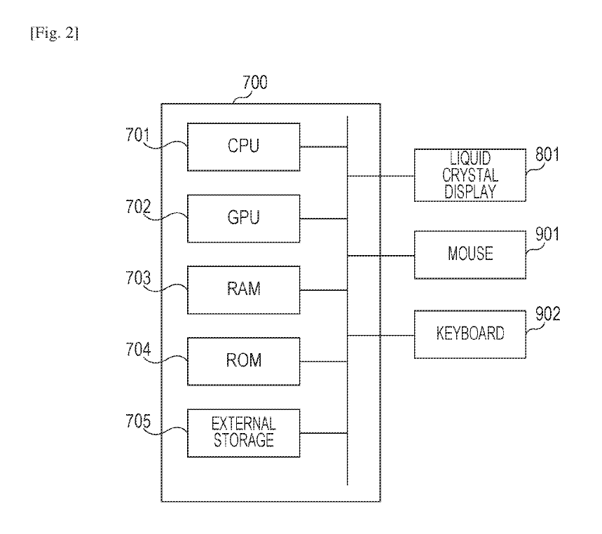

FIG. 2 shows a specific configuration of the computer 700 according to this embodiment. The computer 700 according to this embodiment includes a CPU 701, a GPU 702, a RAM 703, a ROM 704, and an external storage 705. Also, a liquid crystal display 801 serving as a display unit 800, and a mouse 901 and a keyboard 902 serving as the input unit 900 are connected with the computer 700.

### Display Unit 800

The display unit 800 is a display, such as a liquid crystal display or an organic electro luminescence (EL) display. The display unit 800 is a device that displays an image based on, for example, optical coefficient information and specimen information acquired by the computer 700, a numerical value of a specific position, and so forth. The display unit 800 may display GUI for operating the image and device.

### Input Unit 900

The input unit 900 can be configured of, for example, a mouse and a keyboard operable by a user. Alternatively, the display unit 800 may be configured of a touch panel, and the display unit 800 may serve as the input unit 900.

The respective configurations of the photoacoustic apparatus may be configured of respectively different apparatuses, or may be configured of a single integrated apparatus. Alternatively, at least a partial configuration of the photoacoustic apparatus may be configured as a single integrated apparatus.

### Acoustic Matching Material 1100

An acoustic matching material 1100 is described although it is not a configuration of the photoacoustic apparatus. For the acoustic matching material 1100, water, ultrasonic gel, or the like is used. The acoustic matching material 1100 is for allowing an acoustic wave to propagate between the holding unit 300 and the receiving elements 411 to 414. The acoustic matching material 1100 may be a material having a small attenuation in acoustic wave. If irradiation light is transmitted through the acoustic matching material, the acoustic matching material may be transparent to the irradiation light.

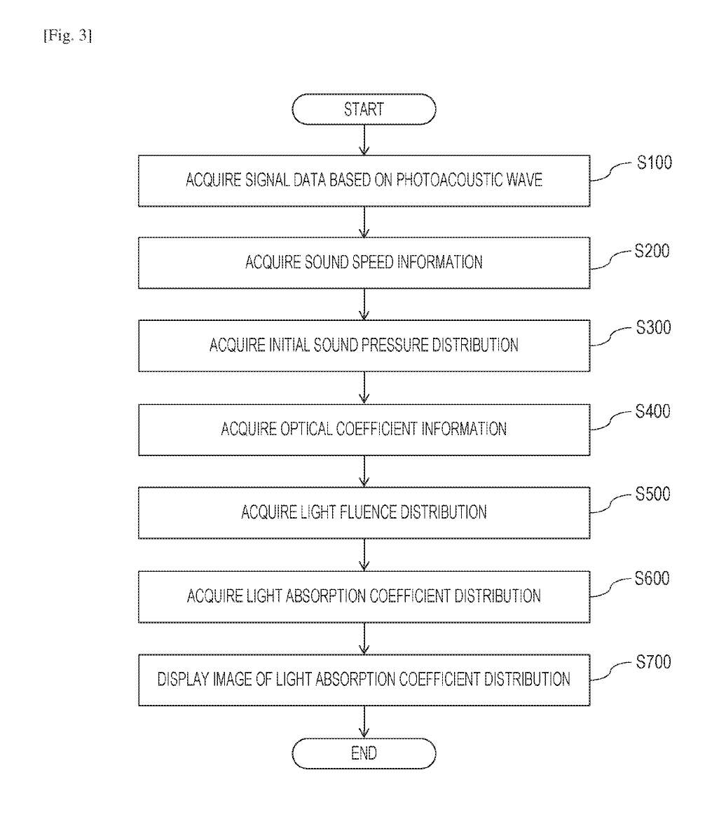

An operation of the photoacoustic apparatus according to this embodiment is described below with reference to a flowchart in FIG. 3.

### S100: Step of Acquiring Signal Data Based on Photoacoustic Wave

The light emitted from the light source 110 is guided by a bundle fiber serving as the optical system 120 to the specimen 1000. The optical system 120 irradiates the specimen 1000 with the light through the holding unit 300. A light absorber in the specimen 1000 absorbs the irradiation light, expands in volume, and generates a photoacoustic wave 1020. This photoacoustic wave 1020 propagates through the specimen 1000 and the acoustic matching material 1100, and reaches the receiving element group 410. The respective receiving elements 411 to 414 receive this photoacoustic wave and output electric signals. Thus, the receiving element group 410 outputs an electric signal group. An electric signal output from a receiving element is a signal in time series representative of a variation with time of the pressure of the photoacoustic wave which has reached the receiving element.

The signal data collecting unit 600 converts the electric signal group being an analog signal group output from the receiving element group 410 into a digital signal group. This digital signal group is stored in the memory 710. That is, signal data based on the photoacoustic wave is stored in the memory 710.

Also, the drive unit 500 may move the receiving unit 400, and the receiving unit 400 may receive the photoacoustic wave at a plurality of different positions. The drive unit 500 includes a motor such as a stepping motor that generates a drive force, a drive mechanism that transmits the drive force, and a position sensor that detects position information of the receiving unit 400. For the drive mechanism, for example, a lead screw mechanism, a link mechanism, a gear mechanism, or a hydraulic mechanism may be used. For the position sensor, for example, an encoder or a potentiometer such as a variable resistor may be used. The drive unit 500 can change the relative position between the specimen 1000 and the receiving unit 400 first-dimensionally, second-dimensionally, or third-dimensionally.

The drive unit 500 may fix the receiving unit 400 and move the specimen 1000 as long as the drive unit 500 can change the relative position between the specimen 1000 and the receiving unit 400. To move the specimen 1000, a configuration is conceivable in which the specimen 1000 is moved by moving the holding unit 300 holding the specimen 1000. Alternatively, the drive unit 500 may move both the specimen 1000 and the receiving unit 400. Also, the drive unit 500 may move the relative position continuously or by step and repeat.

### S200: Step of Acquiring Sound Speed Information

The sound speed acquiring unit 720 acquires sound speed information of the specimen in the propagation path of the acoustic wave. The sound speed acquiring unit 720 may acquire the sound speed information of the specimen by any of known methods.

The sound speed acquiring unit 720 may acquire the sound speed information of the specimen on the basis of the signal level of the signal data based on the photoacoustic wave stored in the memory 710. For example, the sound speed information of the specimen may be acquired on the basis of a variation in the signal level corresponding to a region of interest as described in Japanese Patent Laid-Open No. 2011-120765. Alternatively, the sound speed acquiring unit 720 may acquire the sound speed information of the specimen by analyzing specimen information as distribution information generated from the signal data based on the photoacoustic wave stored in the memory 710.

For example, a region of interest is set, and the region of interest is imaged every time when the sound speed information of the specimen is changed. Then, the sound speed acquiring unit 720 may acquire the sound speed information of the specimen having the maximum brightness value of a pixel value, the maximum contrast, or the maximum sum total of differential values (corresponding to the degree of the edge of the image) with respect to the spatial direction in the region of interest, as the sound speed information of the specimen that optimally images the region of interest. The region of interest may be predetermined, or may be designated by the user using the input unit 900. Also, the image to be evaluated is not limited to an image relating to the initial sound pressure distribution acquired by the initial sound pressure acquiring unit 730, and may be an image relating to the light absorption coefficient distribution acquired by the light absorption coefficient acquiring unit 760. In the latter case, for the optical coefficient information of the specimen 1000 required for acquiring the light absorption coefficient distribution, a temporary value may be previously set. Also, the light absorption coefficient distribution to be evaluated may be acquired by processing of exponentially increasing the gain of the brightness value from the irradiation position with light with respect to the initial sound pressure distribution.

In case where one of these methods of acquiring the sound speed information by using the signal data of the photoacoustic wave is applied to the photoacoustic apparatus, it is not required to perform additional measurement for acquiring the sound speed information. Also, it is not required to add hardware for acquiring the sound speed information.

Still alternatively, the sound speed acquiring unit 720 may acquire the sound speed information of the specimen 1000 on the basis of structure information of the specimen 1000 acquired by a modality other than the photoacoustic apparatus. A typical sound speed in each structure configuring a living body is known. Hence, for example, the structure of each position in the specimen acquired by analyzing an image acquired by another modality apparatus, such as an ultrasound diagnostic apparatus, MRI, or CT may be specified, and the sound speed corresponding to each structure may be allocated.

Yet alternatively, the sound speed acquiring unit 720 may acquire the sound speed information by receiving information input by the user using the input unit 900. For example, the initial sound pressure acquiring unit 730 may acquire the initial sound pressure distribution by a method in S300 (described later) on the basis of predetermined sound speed information, and may cause the display unit 800 to display an image relating to the initial sound pressure distribution. Then, when the user inputs different sound speed information by using the input unit 900, an image corresponding to the similarly input sound speed information can be displayed on the display unit 800. Then, the user can change the sound speed information by using the input unit 900, and input desirable sound speed information as the optimal sound speed information while checking the image displayed on the display unit 800. The sound speed acquiring unit 720 can acquire the optimal sound speed information input by the user.

In a breast as the specimen 1000, the ratio of the mammary gland layer decreases with age and the fat layer becomes dominant as a rough tendency. Typically, the sound speed of the fat layer is in a range from 1422 to 1470 m/s, and the sound speed of the mammary gland layer is in a range from 1510 to 1530 m/s. Hence, it is understood that the sound speed in the specimen 1000 decreases with age. Owing to this, for example, the memory 710 may store a relational expression or a relational table between the age and sound speed information. In this case, when the user inputs information relating to the age by using the input unit 900, the sound speed acquiring unit 720 may read out the sound speed information corresponding to the age information from the memory 710, or may calculate the sound speed information according to the relational expression.

Alternatively, the sound speed acquiring unit 720 may acquire the sound speed information on the basis of temperature information of the specimen 1000. For example, the photoacoustic apparatus may further include a temperature sensor (not shown) that measures the temperature of the specimen 1000. Also, the memory 710 may store a relational expression or a relational table between the temperature and sound speed information of the specimen 1000. The sound speed acquiring unit 720 may acquire the sound speed information of the specimen 1000 according to the relational expression or the relational table stored in the memory 710 on the basis of the specimen 1000 acquired by the temperature sensor.

With the above-described method, the sound speed information of the specimen can be acquired every measurement. If the sound speed information on the same specimen has been acquired before, the previously acquired sound speed information may be read out from the memory 710 and acquired.

The sound speed information of the specimen in this embodiment indicates a representative value of a sound speed in a specimen acquired on the basis of an assumption that the specimen is a uniform medium. That is, the sound speed information of the specimen in this embodiment indicates a representative value of a sound speed acquired when the sound speed at any position of the specimen 1000 is assumed to be constant.

Also, sound speed information of a configuration other than the specimen (the holding unit 300 or the acoustic matching material 1100) included in the propagation path of the acoustic wave may be acquired by the above-describe known method similarly to the sound speed information of the specimen. Also, if the sound speed information of the configuration other than the specimen is already known, the sound speed information may be previously stored in the memory 710, and may be read out from the memory 710 and acquired.

In this embodiment, the representative value of the sound speed of the specimen serves as the sound speed information; however, distribution information representing values of sound speeds at respective positions of the specimen may serve as the sound speed of the specimen as described later in a second embodiment. Alternatively, a representative value of a sound speed acquired on the basis of an assumption that the propagation path of the acoustic wave is entirely a uniform medium may serve as the sound speed information. Still alternatively, since a delay time from a specific position in the specimen to each receiving element is determined on the basis of the propagation speed of the acoustic wave and the distance from the specific position in the specimen to each receiving element, the delay time may serve as the sound speed information.

### S300: Step of Acquiring Initial Sound Pressure Distribution

The initial sound pressure acquiring unit 730 serving as a specimen information acquiring unit acquires the initial sound pressure distribution in the specimen 1000 on the basis of the electric signal group stored in the memory 710, the sound speed information acquired in S200, and the position information of the respective receiving elements 411 to 414. For a method of reconfiguring the initial sound pressure distribution, a known reconfiguration method, such as a time domain reconfiguration method, a Fourier domain reconfiguration method, or a model base reconfiguration method (repetitive reconfiguration method) may be employed. For example, a time domain reconfiguration method called universal back-projection (UBP) as described in Physical Review E71, 016706 (2005) may be employed.

The initial sound pressure acquiring unit 730 may read out the position information of the respective receiving elements 411 to 414 previously stored in the memory 710. Alternatively, the initial sound pressure acquiring unit 730 may acquire the position information of the respective receiving elements 411 to 414 by receiving the position information of the receiving unit 400 from a position sensor included in the drive unit 500 upon light irradiation as a trigger.

### S400: Step of Acquiring Optical Coefficient Information

The memory 710 stores a relational expression or a relational table representing the relationship between the sound speed information and the optical coefficient information. The optical coefficient acquiring unit 740 calculates the optical coefficient information of the specimen on the basis of the sound speed information of the specimen acquired by the sound speed acquiring unit 720 according to the relational expression stored in the memory 710. Alternatively, the optical coefficient acquiring unit 740 reads out the optical coefficient information of the specimen corresponding to the sound speed information of the specimen acquired by the sound speed acquiring unit 720, from the relational table stored in the memory 710.

The optical coefficient information according to this embodiment indicates at least one representative value of a light absorption coefficient μₐ, a reduced scattering coefficient μₛ′, and an effective attenuation coefficient μ_{eff} acquired on the basis of the assumption that the specimen 1000 is a uniform medium. That is, the optical coefficient information of the specimen in this embodiment indicates a representative value of an optical coefficient acquired when the optical coefficient at any position of the specimen 1000 is assumed to be constant.

In this embodiment, the representative value of the optical coefficient of the specimen serves as the optical coefficient information; however, distribution information representing values of optical coefficients at respective positions of the specimen may serve as the optical coefficient information of the specimen as described later in the second embodiment. Alternatively, a representative value of an optical coefficient acquired on the basis of the assumption that the propagation path of the light is entirely a uniform medium may serve as the optical coefficient information.

The optical coefficient information of a configuration other than the specimen may be acquired on the basis of the sound speed information similarly to the optical coefficient information of the specimen. If the optical coefficient information of the configuration other than the specimen is already known, the optical coefficient information may be previously stored in the memory 710, and may be read out from the memory 710 and acquired.

The relationship between the sound speed information and the optical coefficient information is described now. A case where a breast is assumed as the specimen 1000 is described. The main structure of a breast includes fat and mammary glands. It is known that the breast has two layers including a fat layer and a mammary gland layer, and the ratio and distribution of these layers are different depending on the individual. Typically, the sound speed of the fat layer is in the range from 1422 to 1470 m/s, and the sound speed of the mammary gland layer is in the range from 1510 to 1530 m/s. That is, the sound speed decreases if the fat layer increases, and the sound speed increases if the mammary gland layer increases.

The optical coefficient information is affected by the blood present in the fat and the mammary glands. For example, the light absorption coefficient of hemoglobin in blood is largely affected particularly with a wavelength of around 800 nm as compared with the light absorption coefficients of the fat and the mammary glands. Owing to this, even if the blood vessel density per unit volume in a tissue is about 0.1%, a significant different appears with respect to the light absorption coefficients of the fat and the mammary glands. Also, comparing the fat layer and mammary gland layer with each other, typically, the blood vessel density is higher in the mammary gland layer anatomically. Thus, it is conceivable that, in a specimen having the mammary gland layer by a large amount, the sound speed tends to be high, and the light absorption coefficient of the specimen with a near infrared wavelength (around 800 nm) tends to be large. As described above, it is conceivable that the relationship between the sound speed information and the optical coefficient information correlates with the tissue component in the breast.

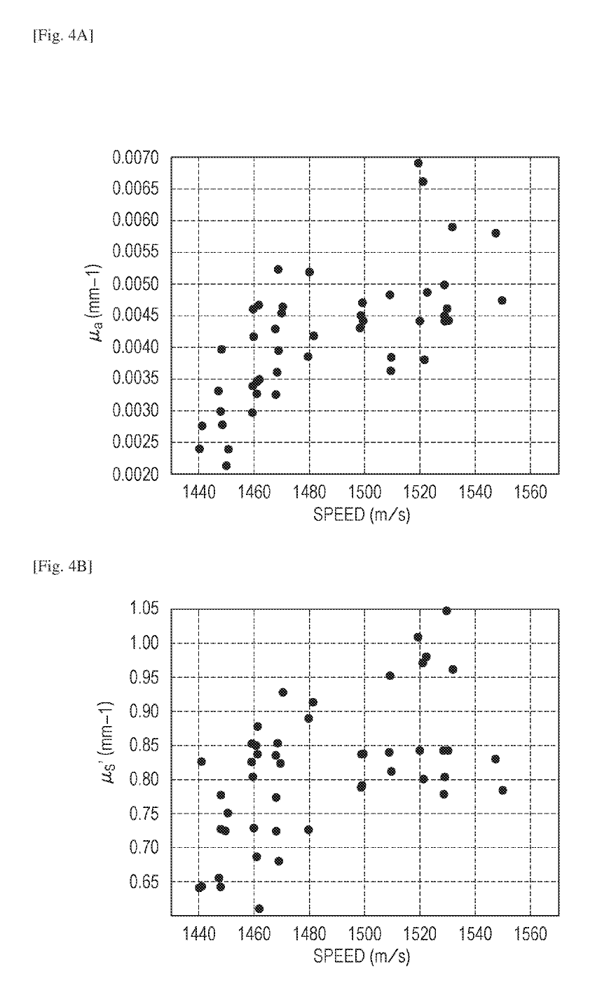

FIGS. 4A and 4B are scatter diagrams each representing the relationship between the sound speed information and the optical coefficient information (μₐ and μₛ′) acquired when the structure of a breast is changed by a simulation. In FIG. 4A, the horizontal axis plots the sound speed, and the vertical axis plots the light absorption coefficient μₐ. In FIG. 4B, the horizontal axis plots the sound speed, and the vertical axis plots the reduced scattering coefficient μₛ′ in the breast. In this calculation, the representative values of the light absorption coefficient and the reduced scattering coefficient were calculated on the basis of an assumption that the tissue component of the specimen was uniform at any position. Also, the calculation was performed while randomly changing the ratio of the mammary gland layer and the fat layer, the temperature of the specimen, the ratio of water in the mammary gland layer, the blood vessel densities in the mammary gland layer and the fat layer, and the oxygen saturation of blood. In this calculation, the ratio of the fat was changed in a range from 30% to 90%, and the ratio of the mammary glands was changed in a range from 10% to 70%. The blood vessel density was changed in a range from 0.1% to 1.1%. The oxygen saturation was changed in a range from 70% to 100%. Also, the amount of red blood cells (hematocrit) in blood was changed in a range of 46%±6%, and the hemoglobin molar concentration was changed in a range of 0.0023876±0.00029 (M/L). The sound speed was calculated by using the statistical value of the sound speed of each structure. The light absorption coefficient t, and the reduced scattering coefficient μₛ′ were calculated by using molar light absorption coefficients and molar reduced scattering coefficients of the fat, mammary glands, water, oxyhemoglobin, and deoxyhemoglobin with respect to the wavelength of 795 nm.

The calculation results as shown in FIGS. 4A and 4B can be stored in the memory 710, as a relational table representing the relationship between the optical coefficient information and the sound speed information. Also, an approximate expression can be obtained from the calculation results as shown in FIGS. 4A and 4B, and can be stored in the memory 710, as a relational expression representing the relationship between the optical coefficient information and the sound speed information. Alternatively, a relational table previously created on the basis of the relational expression may be stored in the memory 710. For example, the relational expression can be obtained by any kind of approximation, such as linear or higher-order function approximation, logarithmic function approximation, or exponential function approximation.

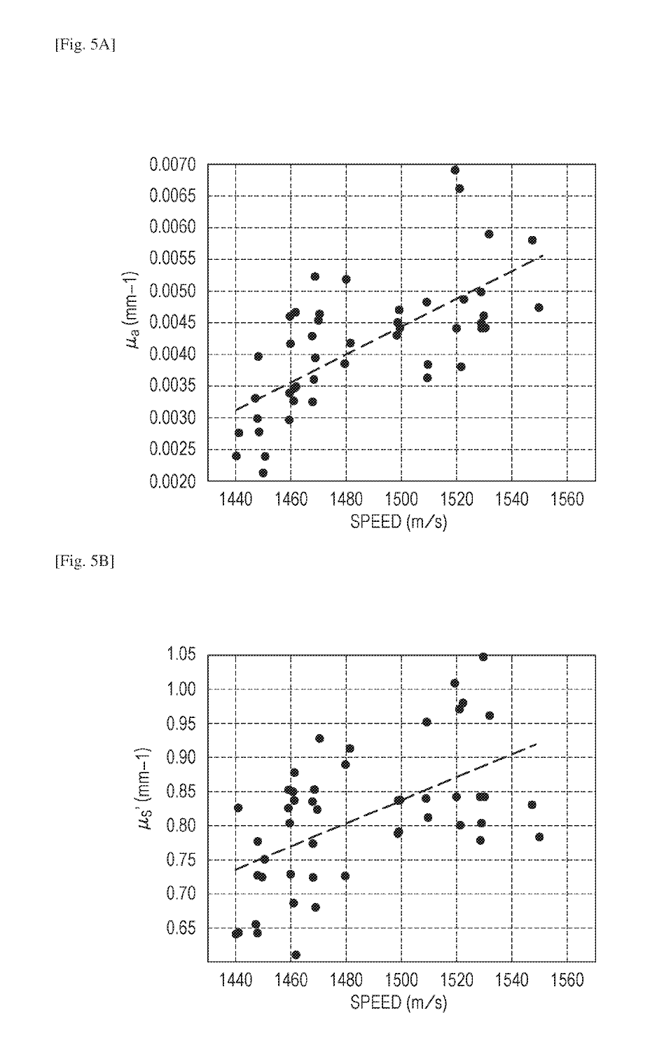

For example, by obtaining a linear function approximate expression by the least square method for the calculation results shown in FIGS. 4A and 4B, approximate expressions (graphs) shown in FIGS. 5A and 5B were acquired. Correlations R between the approximate expressions and calculation results shown in FIGS. 5A and 5B were R=0.6913 for the light absorption coefficient and R=0.5508 for the reduced scattering coefficient. In any case, a significant probability p is 0.000 or lower.

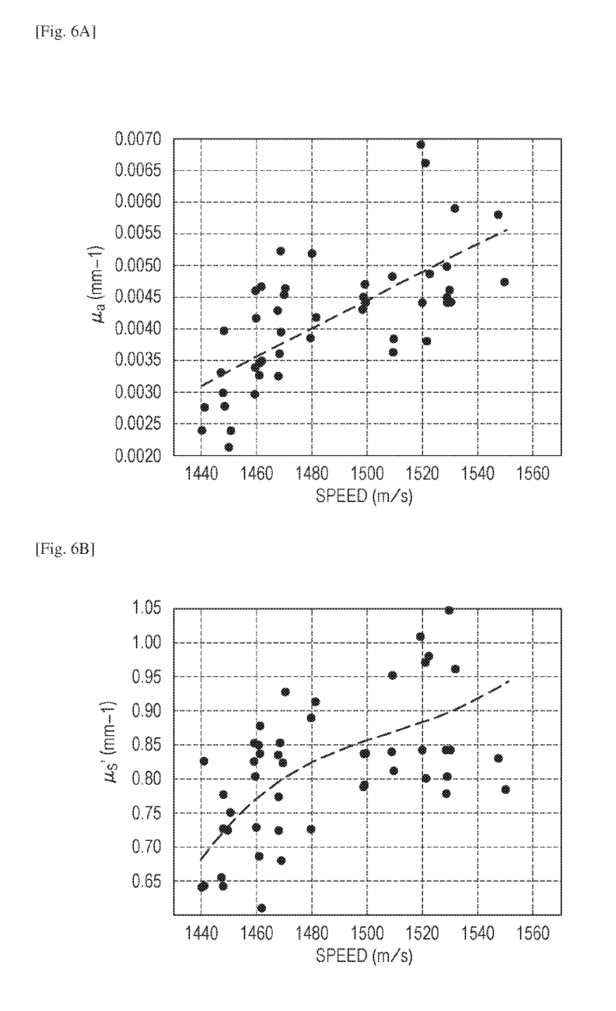

Also, by obtaining a cubic function approximate expression by the least square method for the calculation results shown in FIGS. 4A and 4B, approximate expressions (graphs) shown in FIGS. 6A and 6B were acquired. Correlations R between the approximate expressions and calculation results shown in FIGS. 6A and 6B were R=0.6928 for the light absorption coefficient and R=0.5781 for the reduced scattering coefficient. In any case, a significant probability p is 0.000 or lower.

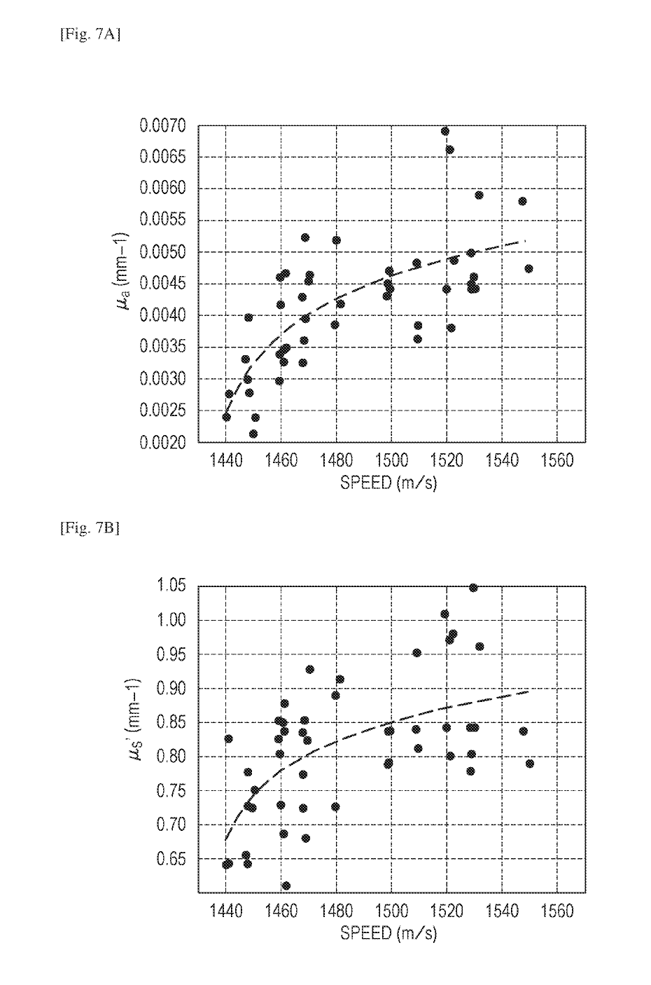

Also, by obtaining a logarithmic function approximate expression by the least square method for the calculation results shown in FIGS. 4A and 4B, approximate expressions (graphs) shown in FIGS. 7A and 7B were acquired. Correlations R between the approximate expressions and calculation results shown in FIGS. 7A and 7B were R=0.7313 for the light absorption coefficient and R=0.5948 for the reduced scattering coefficient. In any case, a significant probability p is 0.000 or lower.

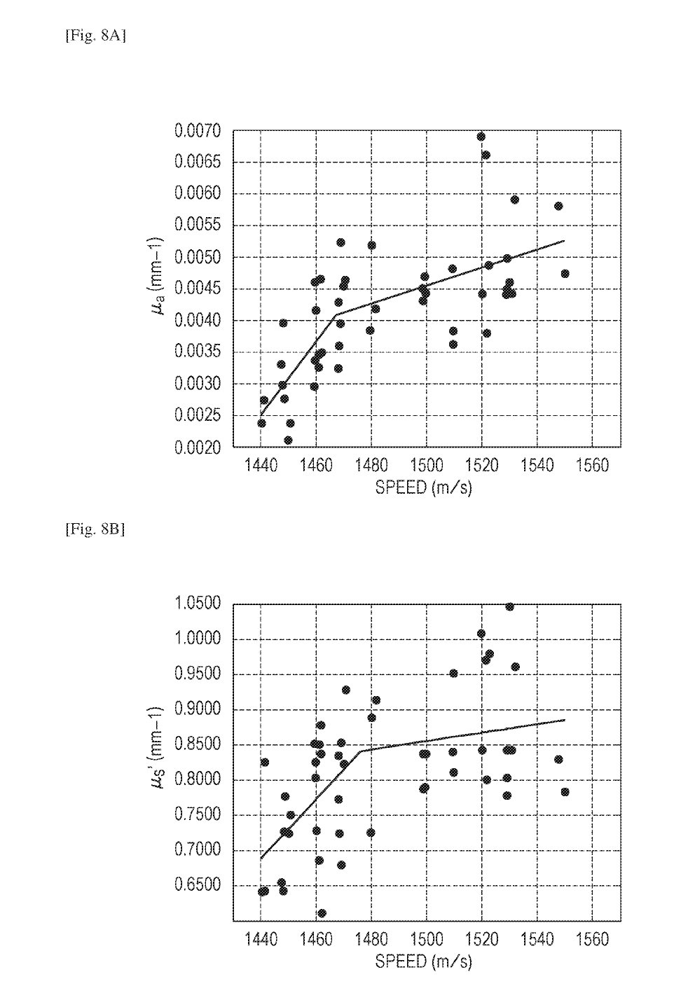

Alternatively, a relational expression in which two linear function approximate expressions are combined may be acquired and stored in the memory 710. For example, if the specimen is a breast, typically, the sound speed of the fat layer is in the range from about 1422 to about 1470 m/s, and the sound speed of the mammary gland layer is in the range from about 1510 to about 1530 m/s. Owing to this, the approximate expression may be switched in a range of the sound speed from 1470 to 1510 m/s. For example, when it is assumed that 1475 m/s is the border between the sound speed of the fat layer and the sound speed of the mammary gland layer, the expression may be divided into two approximate expressions. In this case, for the calculation results shown in FIGS. 4A and 4B, by obtaining a linear function approximate expression in a range of sound speed of 1475 m/s or lower and a linear function approximate expression in a range of sound speed higher than 1475 m/s, approximate expressions (graphs) shown in FIGS. 8A and 8B were acquired. Correlations R between the approximate expressions and calculation results shown in FIGS. 8A and 8B were R=0.7408 for the light absorption coefficient and R=0.5975 for the reduced scattering coefficient. In any case, a significant probability p is 0.000 or lower. As understood from the result, the correlation is higher for the relational expression acquired by the plurality of approximate expressions, as compared with the relational expression acquired by the single approximate expression. As described above, the optical coefficient information may be acquired from the sound speed information by using a relational expression with a high correlation value approximated by a plurality of approximate expressions. The border position of the sound speed may be changed in accordance with the structure of an object to be measured or data of the approximate expression stored in the memory 710 Also, without limiting to the combination of the two linear function approximate expressions, a relational expression in which a plurality of desirable approximate expressions are combined in accordance with a specimen may be used. Alternatively, a plurality of relational expressions and a correlation value or a deviation value with respect to the plurality of relational expressions may be recorded in the memory 710, a relational expression that causes the correlation value to increase or the deviation value to decrease at a sound speed near the sound speed acquired by the sound speed acquiring unit may be selected, and the relational expression may be used for acquiring the optical coefficient information.

In addition to the sound speed and the optical coefficient, a relational table or a relational expression for parameters other than the sound speed, such as the wavelength, tissue density, and acoustic attenuation may be stored in the memory 710. If light with a plurality of wavelengths as described later in a third embodiment is used, since the optical coefficient depends on wavelength, a relational table or a relational expression between the sound speed and the optical coefficient for each wavelength of irradiation light may be prepared. Also, the acoustic attenuation is different depending on the tissue. The acoustic attenuation of fat being 0.6 (dB/MHz/cm) differs from the acoustic attenuation of water being 2.17×10⁻³ (dB/MHz/cm). Hence, for example, by using an ultrasonic CT apparatus as described in J. Acoust. Soc. Am. 131,3802 (2012), both the sound speed and the acoustic attenuation can be measured. A relational table or a relational expression among the sound speed, acoustic attenuation, wavelength, and optical coefficient measured as described above may be acquired and the data thereof may be stored in the memory 710.

Also, if a breast is considered as the specimen, the relational table or the relational expression between the optical coefficient and the sound speed may be changed depending on the density of the mammary gland tissue (mammary gland density). That is, a relational table or a relational expression corresponding to the mammary gland density or the category of the mammary gland density may be saved in the memory 710. Then, the optical coefficient acquiring unit 740 may acquire information relating to the mammary gland density of the specimen, read out the relational table or the relational expression corresponding to the category of the mammary gland density of the specimen to be measured, and acquire the optical coefficient information by using the relational table or the relational expression. The information relating to the mammary gland density is a concept including the mammary gland density or the category of the mammary gland density.

According to Breast Imaging Reporting and Data System (BI-RADS), the mammary gland density is divided into four categories including a. uniform fatty mammary glands, b. scattered fatty mammary glands, c. mammary glands with high mammary gland density, and d. mammary glands with very high mammary gland density. The mammary gland density tends to increase in the order of the categories a, b, c, and d.

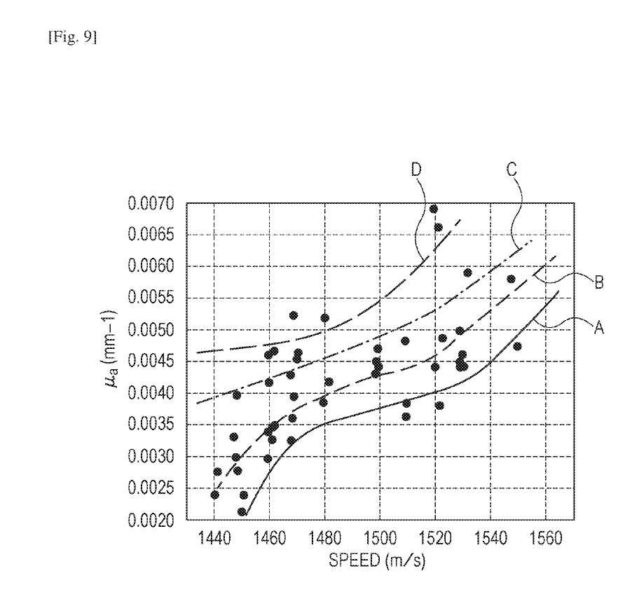

FIG. 9 is a graph representing the relationship between the optical coefficient and the sound speed for each category of mammary gland density with respect to the scatter diagrams shown in FIGS. 4A and 4B. The graphs corresponding to the above-described categories a to d of the mammary gland densities may be conceived as shown in the graphs shown in FIG. 9. A graph A corresponds to the category of a. uniform fatty mammary glands. A graph B corresponds to the category of b. scattered fatty mammary glands. A graph C corresponds to the category of c. mammary glands with high mammary gland density. A graph D corresponds to the category of d. mammary glands with very high mammary gland density.

As shown in FIG. 9, it is conceivable that the sound speed of a specimen with a low mammary gland density is low and the sound speed of a specimen with high mammary gland density is high when substantially equivalent optical coefficients are acquired. Owing to this, the relationship between the sound speed and the optical coefficient is different depending on the category. Owing to this, the sound speed acquiring unit 720 can acquire the sound speed information of the specimen with high accuracy by using the relational expression or the relational table corresponding to the mammary gland density while the mammary gland density serves as a parameter. The input unit 900 may be configured to allow the user to designate the mammary gland density or the category of the mammary gland density. Alternatively, the mammary gland density or the category of the mammary gland density may be estimated on the basis of image data acquired by a modality, such as X-ray mammography, MRI, or CT as a mammary gland density measuring unit. A relational table or a relational expression among the mammary gland density, wavelength, optical coefficient, and sound speed may be stored in the memory 710.

Also, a relational table or a relational expression among a parameter that affects the optical coefficient, such as the age, sex, race, etc., and the optical coefficient and the sound speed may be stored in the memory. The memory may store a relational table corresponding to a plurality of values of respective parameters. The optical coefficient acquiring unit 740 may use at least one of the mammary gland density, wavelength, age, sex, and race as an input parameter in addition to the sound speed, read out a relational table or a relational expression corresponding to the input parameter, and acquire optical characteristic information.

The parameter to be additionally associated with the relational table or the relational expression between the sound speed and the optical coefficient may be designated by the user using the input unit 900. The optical coefficient acquiring unit may acquire the optical coefficient information by using the relational table or the relational expression corresponding to the parameter designated by the user.

Also, the relational table or the relational expression stored in the memory 710 may be re-written.

### S500: Step of Acquiring Light Fluence Distribution

The light fluence acquiring unit 750 serving as a specimen information acquiring unit acquires a light fluence distribution in the specimen 1000 of the light irradiated on the specimen 1000, on the basis of the optical coefficient information acquired in S400. That is, the light fluence acquiring unit 750 acquires the value of light fluence irradiated at each position in the specimen.

The light fluence acquiring unit 750 can acquire the light fluence distribution by a known method on the basis of the optical coefficient information. For example, the light fluence acquiring unit 750 may acquire the light fluence distribution on the basis of parameters, such as an in-plane intensity distribution of the light emitted from the light irradiation unit 100 and the shape of the specimen, in addition to the optical coefficient information. An intensity distribution acquiring unit (not shown) may acquire the in-plane intensity distribution of the light, and a shape acquiring unit (not shown) may acquire the shape of the specimen every measurement. Also, a light quantity meter (power meter) which is not illustrated may measure the total light quantity of the irradiation light. For the calculation method of the light fluence, the finite element method, the Monte Carlo method, etc., may be used. For example, the light fluence distribution may be acquired by a method described in Japanese Patent Laid-Open No. 2011-206192.

### S600: Step of Acquiring Light Absorption Coefficient Distribution

The light absorption coefficient acquiring unit 760 serving as a specimen information acquiring unit acquires a light absorption coefficient distribution on the basis of the initial sound pressure distribution acquired in S300 and the light fluence distribution acquired in S500. The light absorption coefficient acquiring unit 760 divides the initial sound pressure P₀ at each position of the region of interest by the light fluence φ according to Expression (1), and hence can acquire the light absorption coefficient μₐ. On the basis of an assumption that the Gruneisen coefficient r is known, the light absorption coefficient acquiring unit 760 may read out and use the Gruneisen coefficient previously stored in the memory 710 for the calculation.

In this embodiment, the light absorption coefficient distribution acquired in S600 is distribution information representing a value of a light absorption coefficient at each position of the specimen, and differs from the light absorption coefficient acquired on the basis of the assumption that the specimen is a uniform medium in S400. The receiving elements 411 to 414 for the photoacoustic wave have reception band charac-teristics. A reception band characteristic is a reception sensitivity characteristic for the frequency of a photoacoustic wave. The frequency band of a photoacoustic wave is different depending on the size of a light absorber being a generation source of the photoacoustic wave. As the result, a light absorber with a size which generates a frequency that can be received by a receiving element is mainly imaged. For example, when the center frequency of the reception band of a receiving element is 3 MHz and the sound speed of a specimen is 1480 m/s, the size of a light absorber that can be measured by this receiving element is in a range from about 0.370 mm to about 1.48 mm. The size particularly suitable for the measurement is about 0.493 mm. That is, in this case, it is difficult to image a light absorber having a size smaller than 0.370 mm and a light absorber having a size large than 1.48 mm. Hence, the light absorption coefficient distribution acquired by the photoacoustic measurement is a light absorption coefficient distribution having resolution depending on the reception band characteristic.

### S700: Step of Displaying Image of Light Absorption Coefficient Distribution

The control unit 770 transmits data of the light absorption coefficient distribution of the specimen 1000 to the display unit 800, and causes the display unit 800 to display an image of the light absorption coefficient distribution, a numerical value of a specific position in the light absorption coefficient distribution, and so forth. If the specimen information is three-dimensional distribution information, the control unit 770 can cause a tomography image cut along a desirable cross section, a maximum intensity projection (MIP) image, or an image processed by volume rendering. For example, a 3-D image may be displayed in a plurality of different directions. Also, the user may change the inclination, display region, window level, and window width of the displayed image by using the input unit 900 while checking the display on the display unit 800. The control unit 770 may cause the display unit 800 to display the signal data acquired in S100, the sound speed information acquired in S200, the initial sound pressure distribution acquired in S300, the optical coefficient information acquired in S400, or the light fluence distribution acquired in S500. The input unit 900 may be configured to switch ON/OFF of the display of each piece of information. Also, for the display form, for example, superimposition display or parallel display may be employed.

With the photoacoustic apparatus according to this embodiment, the optical coefficient information of the specimen can be acquired on the basis of the sound speed information of the specimen. Also, with the photoacoustic apparatus according to this embodiment, the light absorption coefficient distribution can be acquired by using the optical coefficient information acquired on the basis of the sound speed information.

### Second Embodiment

In the first embodiment, the representative value of the sound speed based on the assumption that the specimen 1000 is a uniform medium is acquired as the sound speed information of the specimen. However, in this embodiment, an example is described in which distribution information representing a value of a sound speed at each position of the specimen 1000 is acquired as sound speed of the specimen. The apparatus configuration according to this embodiment is similar to that of the first embodiment. A portion different from the first embodiment is described below.

The sound speed acquiring unit 720 acquires a sound speed distribution of the specimen 1000 by a known method as described in S200. For example, the sound speed acquiring unit 720 may acquire a sound speed distribution acquired by using an ultrasonic CT apparatus described in J. Acoust. Soc. Am. 131,3802 (2012), as the sound speed information. In this embodiment, since the sound speed information with regard to the sound-speed non-uniformity of the specimen 1000 can be acquired, the initial sound pressure acquiring unit 730 can acquire initial sound pressure information with higher accuracy than the first embodiment, by suing the sound speed distribution of the specimen 1000.

The optical coefficient acquiring unit 740 can acquire a value of an optical coefficient at each position of the specimen 1000 from the value of the sound speed at each position of the specimen 1000, according to the relational expression or the relational table between the sound speed and the optical coefficient stored in the memory 710. That is, in this embodiment, the optical coefficient acquiring unit 740 can acquire an optical coefficient distribution of the specimen 1000 on the basis of the sound speed distribution of the specimen 1000. For example, the optical coefficient acquiring unit 740 replaces the value of the sound speed with the value of the optical coefficient according to the relational expression or the relational table for each position of the specimen, and hence acquires the optical coefficient distribution of the specimen 1000 on the basis of the sound speed distribution of the specimen 1000.

The optical coefficient acquiring unit 740 may perform interpolation processing on the sound speed information acquired by the sound speed acquiring unit 720, and acquire the sound speed information having resolution higher than the resolution of the original sound speed information. Further, the optical coefficient acquiring unit 740 acquires the optical coefficient information on the basis of the sound speed information treated with the interpolation processing, and hence can acquire optical coefficient information having resolution higher than the resolution of the sound speed information acquired by the sound speed acquiring unit 720.

Alternatively, the optical coefficient acquiring unit 740 performs the interpolation processing on the acquired optical coefficient information, and hence can acquire the optical coefficient information with resolution higher than the original resolution determined by the resolution of the sound speed information.

With these methods, even if the sound speed acquiring unit 720 acquires the sound speed information with low resolution, the optical coefficient acquiring unit 740 can acquire the optical coefficient information with high resolution. Accordingly, the optical coefficient information with high resolution can be acquired with a small amount of calculation. For the method of the interpolation processing, any interpolation processing, such as linear interpolation, cubic interpolation, spline interpolation, or nearest point interpolation may be used.

Since light is strongly diffused in a specimen with DOT as described in NPL 1 that can acquire optical coefficient information at each position of the specimen, it has been difficult to acquire optical coefficient information with high resolution. In contrast, with this embodiment, the resolution of the optical coefficient information depends on the sound speed information. Owing to this, with this embodiment, by acquiring the optical coefficient information on the basis of the sound speed information acquired by the method that can acquire the sound speed information with high resolution, the optical coefficient information with resolution higher than related art such as DOT can be acquired. It is known that PAI typically has higher resolution than DOT. Owing to this, the resolution of the optical coefficient information acquired from the sound speed information acquired on the basis of the reception signal of the photoacoustic wave is typically higher than the resolution of the optical coefficient information acquired by DOT.

The light fluence acquiring unit 750 may acquire the light fluence distribution on the basis of the optical coefficient distribution of the specimen acquired by the optical coefficient acquiring unit 740. With this embodiment, since the optical coefficient information with regard to the non-uniformity of the optical coefficient in the specimen is used, the light fluence distribution can be acquired with hither accuracy than the first embodiment.

The shape acquiring unit (not shown) may acquire shape information of the specimen while distinguishing the inside and outside of the specimen on the basis of the sound speed distribution acquired by the sound speed acquiring unit 720. Then, the light fluence acquiring unit 750 may acquire the light fluence distribution on the basis of the shape information of the specimen acquired from the sound speed distribution and the optical coefficient distribution acquired from the sound speed distribution. If the sound speed acquiring unit 720 acquires the sound speed distribution on the basis of the photoacoustic wave, the sound speed acquiring unit 720 can acquire the light fluence distribution with regard to the shape information of the specimen without using additional configuration.

The light absorption coefficient acquiring unit 760 acquires the light absorption coefficient distribution on the basis of the initial sound pressure distribution acquired by the initial sound pressure acquiring unit 730, and the light fluence distribution acquired by the light fluence acquiring unit 750.

The control unit 770 causes the display unit 800 to display an image of the light absorption coefficient distribution, a numerical value of a specific position, and so forth. In this embodiment, the control unit 770 may cause the display unit 800 to display an image of the optical coefficient distribution acquired by the optical coefficient acquiring unit 740, a numerical value of a specific position, and so forth. Also, if only the optical coefficient distribution acquired by the optical coefficient acquiring unit 740 is displayed, the steps in S200 and S400 are executed, and the steps in S100, S300, S500, and S600 may be omitted.

In the optical coefficient distribution, a region with a known optical coefficient because the kind of the portion is previously known or due to other reason may be replaced with a known value regardless of the sound speed value.

The photoacoustic apparatus according to this embodiment can acquire the sound speed distribution of the specimen and acquire the optical coefficient distribution of the specimen from the sound speed distribution of the specimen. Accordingly, the light absorption coefficient distribution can be acquired with higher accuracy than the first embodiment, on the basis of the initial sound pressure distribution acquired with higher accuracy than the first embodiment and the light fluence distribution acquired with further high accuracy.

### Third Embodiment

In this embodiment, an example is described in which spectral information, for example, information relating to the concentration of a substance configuring a specimen is acquired on the basis of a photoacoustic wave generated by irradiating the specimen with light with a plurality of mutually different wavelengths.

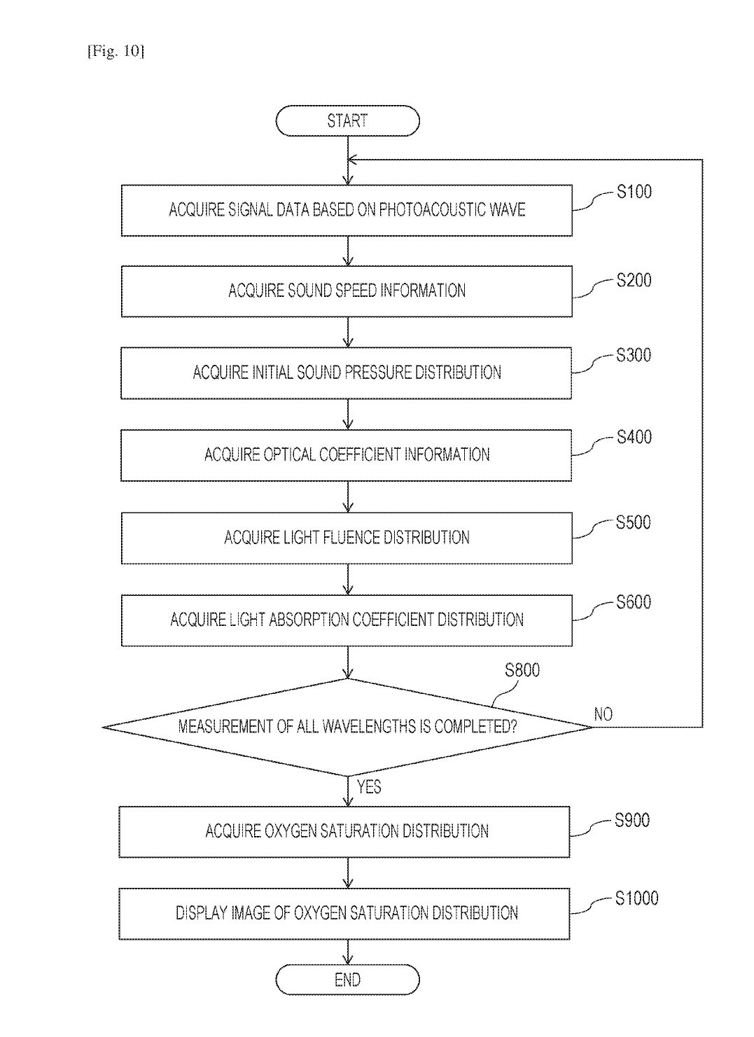

An operation of a photoacoustic apparatus according to this embodiment is described below with reference to a flowchart in FIG. 10. In this embodiment, a photoacoustic apparatus similar to that according to the first embodiment or the second embodiment is used.

In this embodiment, first, steps from S100 to S600 are executed by using light with a first wavelength λ1, and a light absorption coefficient distribution corresponding to the first wavelength is acquired. The control unit 770 determines whether or not the measurement has completed for all wavelengths (S800). If the measurement for all wavelengths is not completed, the control unit 770 changes the wavelength of the light emitted from the light irradiation unit 100, and executes the steps from S100 to S600 again. That is, the steps from S100 to S600 are executed by using light with a second wavelength λ2, and a light absorption coefficient distribution corresponding to the second wavelength is acquired. In this embodiment, the memory 710 stores a relational table or a relational expression between sound speed information and optical coefficient information corresponding to each of the plurality of wavelengths. Then, in S400, the optical coefficient acquiring unit 740 reads out the relational table or the relational expression corresponding to each wavelength from the memory 710, and acquires the optical coefficient information for each wavelength.

Then, the concentration acquiring unit 780 serving as a specimen information acquiring unit acquires an oxygen saturation distribution as information relating to the concentration of a substance configuring the specimen (S900). Hereinafter, an example of a method of acquiring the oxygen saturation distribution is described.

When λ₁ and λ₂ are wavelengths of irradiation light, and ε_{Hb} is a molar light absorption coefficient [1/(mm×M)] of oxyhemoglobin and deoxyhemoglobin,

ε_{HbO}_{₂} [Math.]

and when C_{Hb} is a concentration [M] of each hemoglobin,

C_{Hbo}_{₂} [Math.2]

a light absorption coefficient distribution μₐ corresponding to each wavelength is expressed by Expression (2).

μₐ(λ₁,r)=ε_{Hb}(λ₁,r)·C_{Hb}+ε_{HbO}_{₂}(λ₁,r)·C_{HbO}_{₂} [Math.3]

μₐ(λ₂,r)=ε_{Hb}(λ₂,r)·C_{Hb}+ε_{HbO}_{₂}(λ₂,r)·C_{HbO}_{₂} (2)

An oxygen saturation SO2 is a ratio of the concentration of oxyhemoglobin with respect to the concentration of total hemoglobin, and hence is defined by Expression (3).

$$\begin{matrix}[ Math . 4 ] & \\ SO 2 = \frac{{C}_{{HbO}_{2}} ( r )}{{C}_{{HbO}_{2}} ( r ) + {C}_{Hb} ( r )} & ( 3 )\end{matrix}$$

From Expression (2) and Expression (3), the oxygen saturation SO2 is expressed by Expression (4).

$$\begin{matrix}[ Math . 5 ] & \\ SO 2 = \frac{- {ɛ}_{Hb} ( {λ}_{2} ) {μ}_{a} ( {λ}_{1} , r ) / {μ}_{a} ( {λ}_{2} , r ) + {ɛ}_{Hb} ( {λ}_{1} )}{\begin{matrix}{ɛ}_{{HbO}_{2}} ( {λ}_{2} ) - {ɛ}_{Hb} ( {λ}_{2} ) ) \cdot {μ}_{a} ( {λ}_{1} , r ) / \\ {μ}_{a} ( {λ}_{2} , r ) + {ɛ}_{{HbO}_{2}} ( {λ}_{1} ) - {ɛ}_{Hb} ( {λ}_{1} )\end{matrix}} & ( 4 )\end{matrix}$$

Since the molar light absorption coefficient is known, as it is understood from Expression (4), the concentration acquiring unit 780 can calculate the oxygen saturation distribution on the basis of the light absorption coefficient distribution corresponding to the first wavelength and the light absorption coefficient distribution corresponding to the second wavelength.

Also, if it is assumed that light propagation is planar, the light absorption coefficient ratio in Expression (4) can be obtained by Expression (5).

μₐ( ₁,r)/μₐ(λ₂,r)=P₀(λ₁)φ₀(λ₁)/P₀(λ₂)φ₀(λ₂)×exp(μ_{eff}(λ₁)d(r)−μ_{eff}(λ₂)d(r)) (5)

In this Expression, d is a distance from a light irradiation position (specimen surface), φ₀ is a light fluence at the light irradiation position. In this case, as it is understood from Expression (4) and Expression (5), the oxygen saturation can be acquired from the difference between an equivalent attenuation coefficient with the first wavelength and an equivalent attenuation coefficient with the second wavelength. That is, a relational table or a relational expression between the sound speed information and the optical coefficient information may be stored in the memory 710 while the difference in the equivalent attenuation coefficient between the two wavelengths serves as the optical coefficient information.

In addition to the oxygen saturation, the concentration acquiring unit 780 can acquire data which can be acquired through comparison between data based on different wavelengths, such as the concentration of fat, collagen, water, hemoglobin, glucose, or molecular probe.

The control unit 770 causes the display unit 800 to display an image of the oxygen saturation distribution acquired by the concentration acquiring unit 780, a numerical value of a specific position, and so forth (S1000). An image of the initial sound pressure distribution or the light absorption coefficient distribution may be displayed together with the image of the oxygen saturation distribution.

Since the sound speed information has low wavelength dependency, the sound speed information acquired from the reception signal of the photoacoustic wave corresponding to a partial wavelength of the plurality of wavelengths may be used for acquiring the optical coefficient information corresponding to the residual wavelength. Also, the sound speed information acquired from the reception signal of the photoacoustic wave corresponding to a partial wavelength of the plurality of wavelengths may be used for processing on the reception signal of the photoacoustic wave corresponding to the residual wavelength.

For example, in this embodiment, the sound speed acquiring unit 720 acquires sound speed information on the basis of an electric signal corresponding to the first wavelength. Then, the initial sound pressure acquiring unit 730 may acquire an initial sound pressure distribution corresponding to the second wavelength on the basis of the sound speed information acquired on the basis of the electric signal corresponding to the first wavelength and an electric signal corresponding to the second wavelength. Also, the optical coefficient acquiring unit 740 may acquire optical coefficient information corresponding to the second wavelength according to a relational table or a relational expression corresponding to the second wavelength by using the sound speed information acquired on the basis of the electric signal corresponding to the first wavelength.

Also, when the sound speed information is acquired on the basis of the photoacoustic wave, the sound speed information may be acquired on the basis of the photoacoustic wave generated by light with a wavelength having a smaller difference in molar light absorption coefficient between oxyhemoglobin and deoxyhemoglobin. By selecting such a wavelength, even signals or images from blood vessels having a functional difference, such as an artery and a vein, can be handled similarly to each other.

Also, it is desirable to decrease the interval between measurement of the photoacoustic wave with the first wavelength and measurement of the photoacoustic wave with the second wavelength. If the measurement interval is increased, the specimen may more likely move. If the specimen moves, a shift may be generated in images between the wavelengths, and acquisition accuracy for information relating to the concentration may decrease. Owing to this, after the step in S100 is executed using the light with the first wavelength, the step in S100 may be executed using the light with the second wavelength before the other steps are executed.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-135672, filed Jul. 6, 2015, which is hereby incorporated by reference herein in its entirety.

1. An apparatus comprising:
a first acquiring unit configured to acquire information indicative of a relationship between sound speed information and optical coefficient information;
a second acquiring unit configured to acquire sound speed information of a specimen; and
a third acquiring unit configured to acquire optical coefficient information of the specimen by using the sound speed information of the specimen and the information indicative of the relationship.

2. The apparatus according to claim 1, wherein the second acquiring unit acquires a representative value of a sound speed of the specimen as the sound speed information.

3. The apparatus according to claim 2, wherein the third acquiring unit acquires a representative value of an optical coefficient of the specimen as the optical coefficient information by using the representative value of the sound speed of the specimen and the information indicative of the relationship.

4. The apparatus according to claim 1,
wherein the second acquiring unit acquires sound speed values at a plurality of positions of the specimen as the sound speed information, and
wherein the third acquiring unit acquires optical coefficients at the plurality of positions of the specimen as the optical coefficient information by using the sound speed values at the plurality of positions of the specimen and the information indicative of the relationship.

5. The apparatus according to claim 1, further comprising:
a fourth acquiring unit configured to acquire auxiliary information relating to at least one of mammary gland density, age, sex, and race of the specimen,
wherein the information indicative of the relationship is information indicative of a relationship among the auxiliary information relating to the at least one of the mammary gland density, the age, the sex, and the race, the sound speed information, and the optical coefficient information, and
wherein the third acquiring unit acquires the optical coefficient information of the specimen by using the auxiliary information, the sound speed information of the specimen, and the information indicative of the relationship.

6. The apparatus according to claim 5, further comprising:
an input unit configured to input the auxiliary information,
wherein the fourth acquiring unit acquires the auxiliary information by receiving input information from the input unit.

7. The apparatus according to claim 1, further comprising:
a fifth acquiring unit configured to acquire information relating to a wavelength of the light irradiated on the specimen,
wherein the information indicative of the relationship is information indicative of a relationship among the wavelength of the light, the sound speed information, and the optical coefficient information, and
wherein the third acquiring unit acquires optical coefficient information of the specimen corresponding to the wavelength of the light irradiated on the specimen by using the information relating to the wavelength, the sound speed information of the specimen, and the information indicative of the relationship.

8. The apparatus according to claim 1, wherein the information indicative of the relationship is a relational table or a relational expression indicative of the relationship between the sound speed information and the optical coefficient information.

9. The apparatus according to claim 1, further comprising:
a memory configured to store the information indicative of the relationship,
wherein the first acquiring unit acquires the information indicative of the relationship by reading out the information indicative of the relationship stored in the memory.

10. The apparatus according to claim 1, wherein the third acquiring unit saves the sound speed information of the specimen and the optical coefficient information of the specimen in an associated manner.

11. The apparatus according to claim 1, further comprising:
a sixth acquiring unit configured to acquire a signal originated from an acoustic wave generated from the specimen irradiated with light,
wherein the second acquiring unit acquires the sound speed information of the specimen by using the signal.

12. The apparatus according to claim 1, further comprising:
a sixth acquiring unit configured to acquire a signal acquired by receiving an acoustic wave generated from the specimen irradiated with light; and
a seventh acquiring unit configured to
acquire an initial sound pressure distribution in the specimen by using the signal and the sound speed information of the specimen,
acquire a light fluence distribution in the specimen of the light irradiated on the specimen by using the optical coefficient information of the specimen, and
acquire specimen information relating to a light absorption coefficient distribution in the specimen by using the initial sound pressure distribution and the light fluence distribution.

13. The apparatus according to claim 1, further comprising:
a sixth acquiring unit configured to acquire a signal corresponding to a plurality of wavelengths, the signal which is originated from a photoacoustic wave generated from the specimen irradiated with light with the plurality of wavelengths being mutually different from each other,
wherein the information indicative of the relationship is information indicative of a relationship among information relating to the wavelengths of the light, the sound speed information, and the optical coefficient information,
wherein the second acquiring unit acquires the sound speed information of the specimen by using a signal corresponding to a partial wavelength of the plurality of wavelengths, and
wherein the third acquiring unit acquires optical coefficient information of the specimen corresponding to the plurality of wavelengths by using the sound speed information of the specimen acquired by using the signal corresponding to the partial wavelength and the information indicative of the relationship.

14. The apparatus according to claim 13, further comprising:
a seventh acquiring unit configured to acquire specimen information by using the sound speed information acquired by using the signal corresponding to the partial wavelength, the optical coefficient information corresponding to the plurality of wavelengths, and the signal corresponding to the plurality of wavelengths.

15. The apparatus according to claim 14,
wherein the seventh acquiring unit
acquires an initial sound pressure distribution corresponding to the plurality of wavelengths by using the sound speed information acquired by using the signal corresponding to the partial wavelength, and the signal corresponding to the plurality of wavelengths,
acquires a light fluence distribution corresponding to the plurality of wavelengths by using the optical coefficient information corresponding to the plurality of wavelengths, and
acquires the specimen information by using the initial sound pressure distribution corresponding to the plurality of wavelengths and the light fluence distribution corresponding to the plurality of wavelengths.

16. The apparatus according to claim 12, further comprising:
a light irradiation unit configured to irradiate the specimen with light; and
a receiving unit configured to convert an acoustic wave generated from the specimen irradiated with the light from the light irradiation unit into a signal.

17. (canceled)

18. A method of displaying specimen information acquired by using a signal originated from an acoustic wave generated from a specimen irradiated with light, comprising:
acquiring sound speed information of the specimen; and
displaying optical coefficient information of the specimen corresponding to the sound speed information of the specimen, and the specimen information acquired by using the optical coefficient information of the specimen and the signal.

19. The method according to claim 18, further comprising:
receiving input of the sound speed information of the specimen,
wherein, in the displaying, optical coefficient information of the specimen corresponding to the input sound speed information of the specimen is displayed.

20. A method comprising:
receiving input of sound speed information of a specimen;
receiving input of information relating to at least one of mammary gland density, age, sex, and race of the specimen, and a wavelength of light irradiated on the specimen; and
acquiring optical coefficient information of the specimen corresponding to the input sound speed information of the specimen and the input information.

21. A non-transitory computer-readable storage medium which stores a program causing a computer to execute the method according to claim 20.
